(19) United States
(12) Patent Application Publication
(10) Pub. No.: US 2016/0279358 A1
(43) Pub. Date: Sep. 29, 2016

(54) COMPOSITION FOR USE IN AN INHALATION DEVICE AND INHALATION DEVICE USEFUL THEREFOR

(71) Applicant: AROMASTICK AG, Mauren (LI)

(72) Inventor: Jurg SINGER, Mauren (LI)

(21) Appl. No.: 15/033,821

(22) PCT Filed: Sep. 16, 2014
(86) PCT No.: PCT/EP2014/002491

§ 371 Date: May 2, 2016

(30) Foreign Application Priority Data
Oct. 31, 2013 (EP) 13005165.9
Nov. 12, 2013 (EP) 13192533.1

(51) Int. Cl. A61M 15/08; A61K 36/534; A61K 36/53; A61M 15/00; A61K 36/61; A61K 36/899; A61K 36/185; A61K 36/54; A61K 9/00; A61K 36/752

(52) U.S. Cl. CPC A61M 15/08; A61K 9/0043; A61K 36/534; A61K 36/53; A61K 36/752; A61K 36/61; A61K 36/899; A61K 36/185; A61K 36/54; A61M 15/0036

(57) ABSTRACT

The present invention pertains to the field of the prophylaxis and therapy of diseases, preferably by means of the application of essential oils or oil mixtures by inhalation. The invention relates, in particular, to an especially designed inhalation stick and to a composition suitable therefor based on essential oils.

### CROSS-REFERENCES TO RELATED APPLICATIONS

This application is a National Stage filing of International Application PCT/EP 2014/002491, filed Sep. 16, 2014, claiming priority to EP 13 005 165.9 filed Oct. 31, 2013, and to EP 13 192 533.1 filed Nov. 12, 2013, entitled “COMPOSITION FOR USE IN AN INHALATION DEVICE AND INHALATION DEVICE USEFUL THEREFOR”. The subject application claims priority to PCT/EP 2014/002491, EP 13 005 165.9 and EP 13 192 533.1 and incorporates all by reference herein, in their entirety.

### BACKGROUND OF THE INVENTION

The present invention relates to the field of prophylactic or therapeutic treatment of illnesses by means of preferably inhaled application of essential oils or oil mixtures.

In particular, the present invention relates to a composition for use in the prophylactic and/or therapeutic treatment of illnesses, which comprises at least two essential oils different from one another and is applied by means of an inhalation stick, as well as an inhalation stick containing this composition and the use thereof.

Furthermore, the present invention relates to a specially designed inhalation stick (also referred to synonymously as an inhaler or inhalation device, inhaler stick, inhaler, smelling stick, aroma stick, or the like) according to the preamble of the independent patent claim in this regard, as well as the use thereof. In particular, the present invention relates, according to this aspect of the invention, an inhalation stick having an elongated housing sleeve in particular and a rod-shaped storage means in particular for storing a liquid, which comprises at least one active ingredient and is preferably volatile (i.e., in particular a composition according to the present invention), in particular an essential oil (mixture), wherein the housing sleeve has an inner receptacle space for receiving the storage means.

Similarly, the present invention relates to a method for assembling, in particular for filling such an inhalation stick.

Finally, the present invention relates to the use of the composition according to the present invention for the non-therapeutic, in particular symptomatic application, in particular for use as a food, food ingredient or additive, nutritional supplement, flavor composition, or the like, for a variety of illnesses.

The use of aromatherapies based on essential oils has been fundamentally known from the beginning of the 20th century and is predominantly used for the purposes of relaxation or as a relaxation technique, but increasingly also for the treatment of illnesses. In particular, aromatherapy is seen as an alternative to classical mainstream medicine approaches; however, it is sometimes also used in addition to mainstream medical treatment.

Aromatherapy is predominantly based on the application of essential oils, which contain numerous volatile aromatic substances based on secondary plant substances having different effects on the body. Overall, a variety of different essential oils from different plants exist. Accordingly, different effects can be achieved with the aid of different essential oils or the aromatic substances contained therein. Essential oils can unfold an effect both with respect to the physiology and also with respect to the psychology of persons or patients.

However, it is disadvantageous that essential oils are generally offered as individual oils, but not as combination preparations or complex preparations. The large variety of available essential oils having different effects is incomprehensible as a whole for the patient or consumer and requires the consultation of a specialist or aromatherapist for effective therapy. Overall, concepts hardly exist for providing aromatic oil preparations or compositions, which are already provided ready for use and can be used in a targeted manner for treating individual illnesses.

Carrying out aromatherapies or the application of the essential oils is extraordinarily diverse. One possibility is, for example, to apply the aromatic oils or essential oils directly onto the skin, which is sometimes also linked to (aromatic oil) massages. The direct application of essential oils to the skin does not always result in the desired effect, however. In particular, side effects can occur, such as skin irritation or allergic reactions. In addition, the efficacy is not always optimum or is excessively low in the case of application to the skin.

It is often also provided that an aromatherapy is applied in the manner of a steam bath, i.e., filling a basin with hot water together with essential oils and inhaling the rising vapors, preferably with a hand towel over the head. On the one hand, this method is comparatively uncomfortable to handle, on the other hand, the vapors can enter the eyes, so that the mucosa of the eyes are sometimes irritated. In addition, the risk of scalding from the hot water exists.

Furthermore, aromatherapy can also be performed via the room air, for example, by setting up fragrance lamps, which are charged with the respective aromatic oil or essential oil. This method is often also distinguished by an excessively low efficacy. In addition, it is not always desirable to enrich the entire room or the room air with the aromatic substances of the essential oils. It is also problematic or less practical that such an aromatherapy cannot be performed independently of location.

In addition, the fundamental components of aromatherapy or the aromatherapy per se can be administered via inhalation measures or inhalation devices. In general, inhalation devices having complex apparatuses are used for this purpose. It is also possible to apply essential oils with the aid of so-called inhalation sticks or inhaler sticks. The inhalation sticks known from the prior art are often designed disadvantageously for this purpose or are unsuitable for this purpose, however, as described in detail hereafter.

An inhalation stick generally comprises in its design a storage means made of a compressible material which absorbs liquid, which can be, for example, a nonwoven material or the like, which is impregnated using a liquid which emits a specific active ingredient. For usage, the front part of the inhalation stick, at which the inhalation opening is located, is inserted far enough into the nose of the user or guided thereto that the active ingredients can be absorbed as completely as possible via the nose.

In the inhalation sticks known from the prior art, the storage means is introduced into the housing sleeve of the inhalation stick so it is freely movable. In this way, the impregnated storage means comes permanently into contact with the material of the housing sleeve over a large area. Due to the direct contact with the sleeve, a substantial part of the inhalation means diffuses from the impregnated storage means into the material of the primary container, which generally consists of plastic. These problems result in particular in the case of solutions having essential oils. Such inhalation sticks are therefore sometimes not suitable for aromatherapy based on essential oils. Furthermore, filling the freely moving storage means of the known inhalation stick has proven to be problematic. The storage means is typically impregnated before the insertion. However, hygienic problems and sterility problems can arise due to the subsequent handling of the impregnated storage means up to the insertion into the housing sleeve. Inhalation means, with which the storage means has been impregnated, are often lost during the transportation of the storage means to the housing sleeve. Soiling on the exterior of the housing sleeve can also occur in this case. Precise filling with a defined volume or a defined quantity of the essential oils is also not possible in the case of impregnation in the aromatic solution.

In addition, inhalation sticks of the prior art do not always have optimum delivery or application properties. The sometimes large-area application of the storage means to the housing sleeve is often disadvantageous for optimum release of volatile components.

Overall, an optimization therefore cannot always be achieved using the methods for aromatherapy known from the prior art, both concerning suitable compositions per se and also the corresponding application devices, in particular suitable inhalation sticks.

### BRIEF SUMMARY OF THE INVENTION

The present invention is therefore based on the object of providing active compositions for aromatherapy for the targeted treatment of various illnesses, which at least partially avoid or at least partially mitigates the above-mentioned disadvantages of the prior art.

In particular, the present invention is based in this regard on the object of providing compositions based on essential oils, which are suitable for the effective and targeted treatment of illnesses.

Furthermore, the present invention is based on the object of providing an inhalation stick, which at least partially avoids or at least partially mitigates the above-mentioned disadvantages of the prior art. In particular, the present invention is based in this regard on the object of providing an inhalation stick, which is distinguished by simplified handling in the scope of the manufacturing process and in particular enables simplified or improved filling with a composition based on essential oils. In addition, sterility problems are to be avoided or reduced.

To achieve the above-described object, the present invention proposes, according to a first aspect of the invention, a composition as described herein.

Further subject matter of the present invention corresponding to a second aspect of the invention is additionally an inhalation stick according to the independent claim in this regard, which contains the composition according to the invention.

Still further subject matter according to a third aspect of the invention of the present invention is a specially designed inhalation stick according to the independent claim in this regard; further advantageous embodiments are the subject matter of the dependent claims in this regard.

Similarly, according to fourth and fifth aspects of the invention, the subject matter of the present invention is a method for assembling an inhalation stick according to the invention according to the claims in this regard.

Finally, according to a sixth aspect of the invention, the subject matter of the present invention is the use of the composition according to the invention for non-therapeutic, in particular symptomatic application, in particular for use as a food, food ingredient or additive, nutrition supplement, flavor composition, or the like, for a variety of illnesses, as defined in the relevant use claim.

It is apparent that special designs, embodiments, or the like hereafter, which are only described in conjunction with one aspect of the invention, also apply accordingly with respect to the other aspects of the invention, without this having to be expressly mentioned.

Furthermore, it is to be noted for all below-mentioned relative or percentage, in particular weight-related quantity specifications, that they are to be selected by a person skilled in the art in the scope of the present invention such that in total the respective ingredients, active ingredients, additives, or auxiliary materials or the like always result in 100%. However, this is apparent to a person skilled in the art.

Moreover, a person skilled in the art can deviate from the numeric, range, or quantity specifications listed hereafter with respect to an application or as required by an individual case, without leaving the scope of the present invention.

In addition, all parameter specifications or the like mentioned hereafter can fundamentally be determined or ascertained using standardized or explicitly specified determination methods or, however, using determination methods which are routine to a person skilled in the art.

### BRIEF DESCRIPTION OF THE DRAWINGS

FIG. 1 shows a perspective view of an inhalation stick according to the invention,

FIG. 2 shows a sectional view of the inhalation stick from FIG. 1,

FIG. 3 shows a perspective sectional illustration of a housing sleeve of an inhalation stick according to the invention,

FIG. 4 shows a perspective view of a plug sleeve of the inhalation stick according to the invention,

FIG. 5 shows a further perspective view of the plug sleeve from FIG. 4, and

FIG. 6 shows a perspective sectional illustration of the inhalation stick according to the invention in the closed state without storage means.

### DETAILED DESCRIPTION OF THE INVENTION

The subject matter of the present invention is therefore—according to a first aspect of the invention—a composition which is suitable for use in the prophylactic and/or therapeutic treatment of illnesses, or a composition for use in the prophylactic or therapeutic treatment of illnesses, wherein the composition comprises at least two essential oils different from one another, in particular at least three essential oils different from one another, wherein the essential oils are selected from the group of bergamot oil (Citrus aurantium var. bergamia), cedarwood oil (Cupressus funebris Endlicher), cypress oil (Cupressus sempervirens), eucalyptus oil (Eucalyptus globulus), geranium oil (Pelargonium graveolens), gingergrass oil (Cymbopogon martinii var. sofia), lavender oil (Lavandula angustifolia), marjoram oil (Origanum majorana), mandarin oil (Citrus reticulata), clary sage oil (Salvia sclarea), orange oil (Citrus aurantium var. dulcis), peppermint oil (Mentha piperita), pepper oil (Piper nigrum), rosemary oil (Rosmarinus officinalis), thyme oil (Thymus serpyllum), vetiver oil (Vetiveria zizanoides), juniper berry oil (Juniperus communis L.), cinnamon bark oil (Cinnamomum ceylanicum), lemon oil (Citrus limon), grapefruit oil (Citrus decumana), basil oil (Ocimum basilicum), spruce oil (Picea excelsa) and the mixtures and combinations thereof, and wherein the composition is applied by means of an inhalation stick, preferably applied by inhalation, and/or wherein the composition is provided and/or contained in an inhalation stick, wherein the inhalation stick comprises a storage means, preferably a textile storage means, in its interior, wherein the storage means is provided and/or impinged with the composition.

The term “composition” as used in the scope of the present invention is to be understood very comprehensively and refers not only to compositions for prophylactic or therapeutic treatment purposes, in particular not only pharmaceuticals and medications or medical products, but rather also foods, nutritional supplements, food ingredients or additives, flavor compositions, and homeopathic agents, in particular foods and food ingredients or additives.

Surprisingly, the applicants were able to provide concepts or compositions in the scope of the present invention, which ensure effective and targeted treatment of greatly varying illnesses in an aromatherapy approach. A variety of illnesses of both physiological and also psychosomatic nature can be treated using the compositions according to the invention based on targeted selected combinations of essential oils. In the scope of the present invention, it has therefore been possible to provide application-ready and comfortably applicable complex or combination preparations based on essential oils for the targeted aromatherapy-based treatment of selected illnesses.

The concepts according to the invention for the application of the compositions according to the invention are advantageously distinguished in that no or at least substantially no side effects occur, but outstanding efficacy is nonetheless ensured. The compositions can be brought in high concentration very close to the nose or the receptors of the nasal mucosa, in particular the action location, by the inhalation stick, without direct contact of the skin or mucosa with the essential oils occurring, however. Allergic reactions and skin irritation can be avoided in this manner. Furthermore, as a result of the use of an inhalation stick, the problem does not arise, which occurs with use of aroma lamps, however, specifically that the active ingredients or aromatic materials of the essential oils are distributed in the entire room. Furthermore, due to the targeted application of the composition according to the invention, a habituation to the applied odor, which would be accompanied with a loss of effectiveness, is prevented, as is the case upon application of aromatherapy via the room air.

With regard to the application of the composition according to the invention, the outstanding efficacy thus result in that—without wanting to be restricted in this case to this theory—with the intentional and deep inhalation of the essential oils or the volatile components of the essential oils, targeted binding of the aroma molecules or aromatic materials to the odor receptors in the olfactory mucosa in the nose occurs. Via sensory nerves, which are connected to the receptors via openings in the cribriform plate, the olfactory signals resulting due to the binding or accumulation of the aromatic materials or aroma molecules are relayed to the brain, which in turn results in unfolding of the aromatherapy effect.

In the scope of the present invention, essential oils are understood as highly concentrated plant essences or plant extracts, which can be obtained by means of water steam distillation, scarification, or extraction. Essential oils are distinguished in particular by the volatile properties thereof, which also establishes the outstanding suitability for aromatherapy. Overall, a variety of essential oils based on greatly varying types of plants exist, which accordingly offer manifold properties and possible applications. Depending on the oil, essential oils can contain up to 400 different active ingredients based on secondary plant materials. The secondary plant materials are predominantly mixtures of various terpenes, sesquiterpenes, and aromatic compounds. Terpenes are based on isoprene units, wherein monoterpenes consist of two isoprene units and sesquiterpenes consist of three isoprene units. The active spectrum of essential oils is broadly spread out overall, so that greatly varying effects can be achieved.

It has surprisingly been found in the scope of the present invention that specific combinations of essential oils based on the above functional selection supplement or reinforce one another in the effect thereof such that specific illnesses or clinical pictures can be treated using complex preparations or combination preparations in an extremely targeted and effective manner, without side effects occurring, as is frequently the case with medications of mainstream medicine. In addition, the effect can be further increased by the application of the compositions according to the invention using the inhalation sticks according to the invention, since the binding of the active ingredients to the receptors of the nasal mucosa is optimized.

The composition according to the present invention or the inhalation stick for application of the composition according to the invention can be embodied in manifold ways. Preferred specific embodiments are described hereafter in detail for better comprehension.

With regard to the application of the composition according to the invention, in particular with respect to the absorption and release of the active ingredients of the essential oils, it has proven to be particularly advantageous if the storage means of the inhalation stick is formed as porous or air-permeable to absorb the composition according to the invention.

In this context, it is provided in particular that the storage means comprises a textile material or is formed therefrom, in particular based on cotton, cellulose, or cellulose derivatives, preferably cellulose acetate (for example, storage means based on bonded acetate fiber(s)), glycerin triacetates, and the mixtures and combinations thereof. The above-mentioned materials and the porosity or air permeability of the storage means enable particularly efficient absorption of the composition according to the invention in the storage means, wherein premature evaporation can also be prevented in particular. The storage life of the compositions according to the invention or the application-ready inhalation sticks can also be improved in particular by the material selection.

In addition, it is preferred according to the invention if the storage means has a water absorption capacity of at least 3 g/g, in particular at least 4 g/g, preferably at least 5 g/g (quantity or weight of water per quantity or weight of storage means). It has proven to be particularly advantageous according to the invention if the storage means has an increased water absorption capacity, so that large quantities of the composition according to the invention can be introduced into the storage means, which in turn further improves the storage life or durability of the application-ready inhalation sticks.

In addition, it is particularly provided according to the invention that the storage means is in the form of a cylinder or rod, in particular having lengths of 10 to 100 mm or thicknesses (diameters) of 1 to 20 mm.

With respect to the storage means, it is additionally preferred if it has a weight of 0.1 to 10.0 g, in particular 0.2 to 7.5 g, preferably 0.3 to 5 g. In this case, the weight specifications relate in particular to the uncharged state of the storage means.

With regard to the quantity of the composition according to the invention in the inhalation stick or the storage means, it can thus vary in broad ranges. However, it is preferable according to the invention if the inhalation stick or the storage means contains the composition in effective, in particular therapeutically effective quantities.

Particularly good efficacy, in particular with regard to the release of the essential oils, is ensured in particular if the inhalation stick and/or the storage means contains the composition in quantities of 0.01 to 10.0 mL, in particular 0.1 to 5 mL, preferably 0.2 to 1 mL.

Similarly, it can be provided that the inhalation stick or the storage means contains the composition in quantities of 0.005 to 15 g, in particular 0.05 to 7.5 g, preferably 0.01 to 1.5 g, particularly preferably 0.1 to 1 g. It can also be provided in the scope of the present invention that the inhalation stick and/or the storage means contains the composition in relative quantities of 0.1 to 300 wt. %, in particular 0.2 to 150 wt. %, preferably 0.5 to 100 wt. %, particularly preferably 1 to 80 wt. %, in relation to the storage means.

With regard to the application of the composition according to the invention, it is thus typically provided in the scope of the present invention that the storage means is capable of releasing the composition in effective, in particular therapeutically effective quantities upon application, in particular inhalation application. In other words, it is provided according to the invention that the composition according to the invention is nasally applied in particular, i.e., the inhalation stick is held directly in front of the nose or is inserted with the tip into the nose. In this manner—as also explained hereafter in detail—uncomplicated treatment, which is at least substantially free of side effects, of greatly varying illnesses can be ensured, because the essential oils do not come into contact with the skin or mucosa. In addition, the effectiveness is improved in relation to the known methods for aromatherapy, because the active ingredients are brought directly into the vicinity of the receptors of the nasal mucosa, but without this resulting in direct contact of essential oil and nasal mucosa, which would in turn result in (mucosal) skin irritations. The composition according to the invention is therefore particularly applied nasally or intranasally. An application via the mouth is also similarly possible, so that the composition according to the invention can also be administered orally or perorally, for example.

Especially with regard to the illnesses to be treated, it has been shown in the scope of the present invention that, using targeted selected combinations of essential oils based on the above-mentioned selection, particularly efficient treatment of illnesses is possible, which are selected from cardiovascular illnesses (illnesses of the cardiac/circulatory system), in particular high blood pressure (hypertonia); infections, in particular colds, rhinitis, and bronchitis; metabolic illnesses, in particular diabetes or obesity; psychosomatic illnesses, in particular stress, physical-mental strain, withdrawal syndromes, and sleep disturbances; and/or chronic pain.

The above-mentioned illnesses thus also relate in particular to so-called “widespread diseases”, which occur particularly frequently in the population and have a corresponding demand for treatment or therapy. In the scope of the present invention, it was completely surprising that the symptoms of the above-mentioned systemic illnesses in particular could be significantly relieved on the basis of nasal application in particular of the compositions according to the invention, without noteworthy side effects occurring. In this context, both physical symptoms, such as excessively high blood pressure, pain, tachycardia, appearances of information, and the like, and also mental or psychosomatic difficulties, such as exhaustion, nervousness, restlessness, tension, and the like can be treated.

In particular, it was surprising in the scope of the present invention that the pharmacodynamics may be positively influenced or accelerated by application of the compositions according to the invention. Surprisingly, it has been shown that upon the use of compositions according to the invention as an accompanying measure, for example, for pain therapy, the latency (i.e., the time until the beginning of action of a pain tablet, for example) and the time until the maximum achievable relief of symptoms (for example, the time until the complete or maximum freedom from pain) can be shortened.

In this context, reference has already been made to the effectiveness studies carried out by the applicant, which impressively prove the surprising efficacy of compositions set in a targeted manner according to the invention with respect to specific illnesses.

With regard to the physicochemical parameters of the compositions according to the invention, these are described in detail hereafter. The determination of the parameters is performed on the basis of DIN-standardized measurement methods and methods which are known to a person skilled in the art and therefore do not require further explanations at this point.

With respect to the density of the composition according to the invention, at 20° C. and atmospheric pressure (101,325 Pa) it is thus preferably in the range of 0.5 to 1.5 g/cm³, in particular in the range of 0.6 to 1.2 g/cm³, preferably in the range of 0.7 to 1.1 g/cm³.

In addition, it is provided according to the invention that the composition is liquid or in the liquid state at 20° C. and atmospheric pressure (101,325 Pa).

Furthermore, the composition according to the invention typically has an index of refraction at 20° C. in the range of 1.300 to 1.700, in particular in the range of 1.350 to 1.650, preferably in the range of 1.400 to 1.600.

In addition, the composition according to the invention is distinguished by an extremely low proportion of volatile organic compounds. It is thus provided according to the invention that the composition has a proportion of VOC (volatile organic compounds) of less than 1 wt. %, in particular less than 0.5 wt. %, preferably 0 wt. % in relation to the composition. The composition according to the invention is therefore at least substantially free of harmful organic substances, so that no side effects linked thereto occur.

With regard to obtaining the composition according to the invention, this is performed by means of production methods known for essential oils. It is provided in particular according to the invention that the composition is obtained by means of compression and/or water steam distillation, in particular from the corresponding starting drugs and/or plant parts, such as fruit peels, fruits, wood, leaves, branches, twig tips, flowers, inflorescences, grass, herbs, roots, rinds, or the like.

Furthermore, it is preferable according to the invention for the composition to be volatile. It is particularly preferable in this context if the composition has a vapor pressure at 20° C. greater than 20 mbar, in particular greater than 24 mbar, preferably greater than 28 mbar, more preferably greater than 30 mbar, particularly preferably greater than 40 mbar, very particularly preferably greater than 60 mbar, still more preferably greater than 100 mbar. The mode of action of the composition according to the invention is accordingly based in the volatile properties, since the aromatherapy effect first arises or the volatile aromatic materials can reach the action locations thereof after application or inhalation only by way of a correspondingly high vapor pressure.

It has proven to be particularly advantageous according to the invention if the composition comprises at least one of the following combinations of essential oils:
(i) peppermint oil/rosemary oil/lavender oil, in particular in weight-related ratios of 40-80/10-50/1-20, preferably for the prophylactic and/or therapeutic treatment of chronic pain;
(ii) grapefruit oil/peppermint oil, in particular in weight-related ratios of 30-70/30-70, preferably for the prophylactic and/or therapeutic treatment of metabolic illnesses, in particular diabetes or obesity;
(iii) eucalyptus oil/peppermint oil/thyme oil, in particular in weight-related ratios of 60-90/5-20/5-20, preferably for the prophylactic and/or therapeutic treatment of infections, in particular colds, rhinitis, and bronchitis;
(iv) lavender oil/vetiver oil/mandarin oil, in particular in weight-related ratios of 20-50/20-50/15-40, preferably for the prophylactic and/or therapeutic treatment of cardiovascular illnesses (illnesses of the heart/circulatory system), in particular high blood pressure (hypertonia), and/or psychosomatic illnesses, in particular stress, physical-mental strain, withdrawal syndromes, and sleep disturbances;
(v) lemon oil/peppermint oil/rosemary oil/grapefruit oil/pepper oil/basil oil, in particular in weight-related ratios of 15-30/15-30/15-30/10-25/2-10/1-8;
(vi) bergamot oil/lemon oil/cedarwood oil/clary sage oil/vetiver oil, in particular in weight-related ratios of 25-40/20-35/10-20/10-20/5-20, preferably for the prophylactic and/or therapeutic treatment of cardiovascular illnesses (illnesses of the heart/circulatory system), in particular high blood pressure (hypertonia), and/or psychosomatic illnesses, in particular stress, physical-mental strain, withdrawal syndromes, and sleep disturbances;
(vii) peppermint oil/vetiver oil/juniper berry oil/thyme oil, in particular in weight-related ratios of 25-45/20-40/20-40/1-15;
(viii) peppermint oil/cypress oil/geranium oil/gingergrass oil, in particular in weight-related ratios of 40-60/25-45/10-30/1-10;
(ix) peppermint oil/rosemary oil/cinnamon bark oil, in particular in weight-related ratios of 65-95/1-15/1-10, preferably for the prophylactic and/or therapeutic treatment of cardiovascular illnesses (illnesses of the heart/circulatory system), in particular high blood pressure (hypertonia), and/or psychosomatic illnesses, in particular stress, physical-mental strain, withdrawal syndromes, and sleep disturbances;
(x) peppermint oil/grapefruit oil/rosemary oil, in particular in weight-related ratios of 75-95/1-12/1-5;
(xi) bergamot oil/marjoram oil/lavender oil/orange oil, in particular in weight-related ratios of 30-50/10-30/10-30/10-30;
(xii) rosemary oil/peppermint oil/basil oil, in particular in weight-related ratios of 40-60/35-55/1-15.

Reference is also made in this context to the following exemplary embodiments, which impressively proves the efficacy of the above-mentioned active ingredient combinations.

Therefore, concepts or compositions are provided in the scope of the present invention, which enable, in a completely surprising manner, targeted and efficacious treatment of various illnesses based on aromatherapies. The nasal application in particular of the compositions according to the invention is particularly simple to handle and is additionally distinguished by its extremely minor side effects. The outstanding efficacy of the composition according to the invention in the treatment of the above-mentioned illnesses is also impressively proved on the basis of the exemplary embodiments and application studies carried out by the applicant as described hereafter.

Further subject matter of the present invention is—according to a second aspect of the invention—an inhalation stick for the inhalation application of active ingredients, wherein the inhalation stick comprises in its interior a storage means, preferably a textile storage means, wherein the storage means is provided and/or impinged with a composition for the prophylactic and/or therapeutic treatment of illnesses, wherein the composition comprises at least two essential oils different from one another, in particular at least three essential oils different from one another, wherein the essential oils are selected from the group of bergamot oil (Citrus aurantium var. bergamia), cedarwood oil (Cupressus funebris Endlicher), cypress oil (Cupressus sempervirens), eucalyptus oil (Eucalyptus globulus), geranium oil (Pelargonium graveolens), gingergrass oil (Cymbopogon martinii var. sofia), lavender oil (Lavandula angustifolia), marjoram oil (Origanum majorana), mandarin oil (Citrus reticulata), clary sage oil (Salvia sclarea), orange oil (Citrus aurantium var. dulcis), peppermint oil (Mentha piperita), pepper oil (Piper nigrum), rosemary oil (Rosmarinus officinalis), thyme oil (Thymus serpyllum), vetiver oil (Vetiveria zizanoides), juniper berry oil (Juniperus communis L.), cinnamon bark oil (Cinnamomum ceylanicum), lemon oil (Citrus limon), grapefruit oil (Citrus decumana), basil oil (Ocimum basilicum), spruce oil (Picea excelsa), and the mixtures and combinations thereof.

Surprisingly, it has been found by the applicant that the efficacy of active ingredients based on essential oils can be significantly improved using the inhalation stick according to the invention. Without wishing to be restricted to this theory in this case, the application of the inhalation stick according to the invention improves the application or administration of the composition according to the invention, in particular also based on a fluidic optimization of the inhalation stick with resulting improved delivery of the composition, which therefore also improves the binding of the active ingredients or aromatic materials to the receptors in the nasal mucosa, in particular with regard to optimized concentration of the active components act the action location. In addition, premature evaporation of the active ingredients from the inhalation stick or into the material of the inhalation stick is avoided. In addition, the inhalation stick according to the invention is extremely comfortable to handle and can be carried along by the patient at any time, since it only has a small size. In contrast to the known methods or measures for aromatherapy, for example, fragrance lamps or aromatic oil massages, the inhalation stick according to the invention can also be applied independently of location.

In this context, it can be provided in particular that the inhalation stick is an inhalation stick as described according to the third aspect of the invention and which contains an above-described composition according to the present invention.

For more extensive details on this aspect of the invention—to avoid unnecessary repetitions—reference can be made to the above statements on the first aspect of the invention, which apply accordingly with respect to the inhalation stick according to the invention.

Further subject matter of the present invention is additionally—according to a third aspect of the present invention—an inhalation stick having an elongated housing sleeve in particular and a rod-shaped storage means in particularly for the reversible storage in particular (in particular reversible absorption) and/or for the delivery or administration of at least one, preferably volatile liquid comprising an active ingredient, in particular an essential oil, or an above-defined composition, wherein the housing sleeve has an inner receptacle space for receiving the storage means, wherein at least one fixing projection, which protrudes in particular in the radial direction into the receptacle space, is provided in the interior in the housing sleeve for the friction-locked fixing of the storage means in the housing sleeve.

In the scope of the present invention, reversible storage, in particular reversible absorption, is to be understood in particular such that the storage means, which is provided or impinged with the preferably volatile liquid, in particular the composition according to the invention, is capable, upon application of the inhalation stick, of releasing or delivering the in particular volatile active ingredients or ingredients contained in the composition according to the invention, so that they can unfold the aromatherapy effect thereof.

The object described at the outset is at least substantially achieved for an inhalation stick of the above-mentioned type according to the invention in that at least one fixing projection, which protrudes in particular in the radial direction into the receptacle space, is provided in the interior in the housing sleeve for the friction-locked fixing of the storage means in the housing sleeve. Various substantial advantages result due to the fixing projection, which protrudes in the radial direction into the receptacle space. Firstly, the clear diameter inside the receptacle space is reduced by the fixing projection protruding in the radial direction into the receptacle space. The clear diameter of the receptacle space which is reduced by the fixing projection is smaller than the external diameter of the rod-shaped storage means, so that after the insertion of the storage means into the housing sleeve, a friction lock results between the fixing projection and the storage means. In this manner, secure fixing and location-stable arrangement of the storage means in the housing sleeve results. It is not only ensured by the location-stable arrangement of the storage means in the housing sleeve that the storage means does not move in the housing sleeve during the handling of the inhalation stick, which is perceived as unpleasant by the user. In addition, it is possible to firstly introduce the uncharged storage means into the housing sleeve and to fill it only subsequently, since a defined and fixed arrangement of the storage means in the housing sleeve is ensured and therefore it is not a concern that the liquid to be introduced into the storage means will be introduced at a point in the housing sleeve at which no storage means is located. This finally also has the result that not only is very exact dosing of the inhalation liquid possible, but hygiene and sterility problems are also avoided. Finally, it is ensured as a result of the formation provided according to the invention of the inner structure of the housing sleeve that the storage means only presses in regions against the inner wall of the receptacle space, so that the diffusion problems only occur to a minor extent in the inhalation stick according to the invention.

With regard to the filling of the storage means with the preferably volatile liquid, in particular the composition according to the present invention, the fixing according to the invention of the storage means is additionally advantageous in that it enables the active ingredient solution (“essential oils”) based on the preferably volatile liquid to be introduced directly into the storage means. In the prior art, non-targeted application or “pouring on” from above onto the storage means often takes place, which has the result that a part of the impregnation solution is not absorbed rapidly enough by the storage means or is distributed improperly in the sleeve in another manner.

While it is already fundamentally sufficient to provide only one fixing projection for fixing the storage means and for spacing it apart at least in regions from the inner wall of the receptacle space, it has proven to be particularly advantageous to provide a plurality of fixing projections in the housing sleeve. They are preferably formed such that the storage means, the diameter of which is to be adapted to the dimensions of the fixing projections, exclusively presses against the fixing projections, without pressing against the remaining inner wall region of the receptacle space. Predominant freedom from contact thus results, so that diffusion cannot occur at these points.

In a particularly advantageous embodiment of the invention, the storage means is arranged centered in the receptacle space by fixing projections which are in particular spaced apart uniformly around the circumference of the receptacle space. A location-defined, central arrangement of the storage means in the housing sleeve results due to the centered arrangement via the fixing projections, so that it is possible to introduce the inhalation liquid via a central nose opening of the housing sleeve into the storage means.

It has proven to be particularly advantageous in this context for the fixing projections to be formed as elongated ribs. A linear contact between the storage means and the fixing projections results due to the rib-like formation of the fixing projections, which improves the friction-locked connection and therefore the location-secure arrangement of the storage means in the housing sleeve. This is significant in particular during the filling procedure. However, it is to be noted that it is also fundamentally possible to provide a plurality of individual projections arranged one behind another in the sleeve longitudinal direction instead of individual elongated ribs.

Finally, however, it has proven to be particularly advantageous to provide three elongated ribs in the housing sleeve, which are each spaced apart from one another by 120°, distributed around the circumference of the receptacle space on the inner wall thereof, to achieve good fixing and centering of the storage means, on the one hand, and to enable only minor diffusion into the housing wall, on the other hand.

The housing sleeve itself, which preferably consists of a plastic, has a front sleeve region and a rear sleeve region. The front, elongated sleeve region is used as an inhalation region, while the rear sleeve region is used as a handle region and therefore for holding the inhalation stick during handling. Such a housing sleeve may be produced relatively simply and cost-effectively, wherein the rear sleeve region should have a somewhat larger external diameter than the front sleeve region for handling reasons.

In one design of the housing sleeve, at least one, preferably a single central nose opening which leads into the receptacle space, is provided at the front end of the front sleeve region. In addition, also providing at least one lateral opening, which leads radially into the receptacle space in particular, on the front sleeve region, but sufficiently spaced apart from the front end, suggests itself. This design has proven to be advantageous from a handling aspect for two reasons. During the handling of the inhalation stick, the front end of the front sleeve region is inserted into the nose of the user or positioned in the region of the nose (in particular in the region of the nostrils). The active ingredients can then be received via the nose opening of the inhalation stick. The spacing of the lateral opening apart from the front nose opening is to be such that the lateral opening is located outside the nose of the user in the inserted state. It is ensured in this way that sufficient air can be inhaled via the lateral opening during the usage of the inhalation stick. Moreover, it is ensured by the central arrangement of the nose opening and the simultaneous central arrangement of the storage means in the housing sleeve that a defined supply of the inhalation liquid or the composition according to the invention to the storage means is possible via the nose opening. In addition, the release of the compositions is improved by the spacing of the storage means apart from the wall of the inhalation stick, which, in particular accompanied by fluidic advantages (i.e., low flow resistance or low pressure loss), results in overall improved administration of the composition according to the invention.

In particular to ensure a location-stable arrangement of the storage means in the housing sleeve for the charging of the storage means, in a preferred embodiment of the invention, it is provided that the ribs in the housing sleeve are provided in the region of the front end of the front sleeve region. In this case, the ribs can finally extend from the front end wall of the housing into the receptacle space in the axial direction.

To additionally ensure a location-defined arrangement of the storage means in the housing sleeve, the ribs each have a stop for the terminal contact of the storage means. It is ensured by the stop in the region of the ribs that the storage means can only be inserted into the housing sleeve up to a specific point, namely the stop point.

In this context, it can moreover be provided that the ribs are each provided with a terminal intake bevel, to ensure the insertion without problems of the rod-shaped storage means into the housing sleeve and the pushing of the storage means onto the ribs for friction-locked holding until reaching the stop.

The housing of the inhalation stick according to the invention is preferably constructed in at least two parts and has, in addition to the housing sleeve, a plug or closure, which is preferably formed as a plug sleeve insertable or inserted into the rear sleeve region. A cap or another cover would also be sufficient per se for the closure of the housing sleeve. The inhalation stick according to the present invention cannot be readily opened by the applier or user due to the above-mentioned approach of a plug, so that the storage means cannot be removed, improperly used or filled, or soiled. However, it has proven to be particularly preferable to use a plug sleeve, on which fixing projections are also provided for the terminal fixing of the storage means. The fixing projections are then preferably to be provided with terminal intake stops. This embodiment makes it substantially easier to assemble the inhalation stick according to the invention. It is thus possible to firstly insert the storage means into the housing sleeve, without contact already having to occur of one end of the storage means with the stops on the ribs provided in the interior in the housing sleeve. The storage means is pressed into its final assembly position via the intake stops by putting on the plug sleeve, so that subsequently, at both ends of the storage means, location-secure holding and fixing results, on the one hand, via the fixing means of the housing sleeve and, on the other hand, via the fixing means on the plug sleeve.

To achieve a simple connection between the plug sleeve and the housing sleeve, it is provided that the two components are latched with one another. In one preferred embodiment, a circumferential catch projection is provided on the outside of the plug sleeve and a circumferential groove or step, under which the catch projection engages, is provided on the inside of the rear sleeve region.

To avoid undesired active ingredient escape from the inhalation stick when it is not in use, a closure cap is provided to be put on the front sleeve region. The closure cap is designed in this case such that the nose and lateral openings are closed in the put-on state. In a simple design, an external thread for screwing the closure cap on to the housing sleeve is provided at the transition from the front sleeve region to the rear sleeve region. An internal thread of the closure cap corresponds to the external thread on the housing sleeve, wherein the screw connection is designed as a whole so that an at least substantially airtight closure and fixed holding of the closure cap on the housing sleeve already takes place after one rotation of the closure cap in relation to the housing sleeve of less than 360°, in particular between 180° and 330°.

It is expressly noted that all above-mentioned intervals also include all intermediate intervals and, moreover, all individual values which lie within the interval limits, and these are considered to be essential to the invention, even if these individual values or intermediate intervals are not specified in detail.

As previously noted, it is provided in particular according to the invention that the inhalation stick contains a composition according to the invention, as defined above, and/or a storage means as described above in conjunction with the first aspect of the invention. The storage means is preferably impinged or provided with a preferably volatile liquid, in particular the composition according to the invention.

Similarly, it can be provided according to the invention that the storage means of the inhalation stick according to the invention is impinged and/or provided with a preferably volatile liquid, in particular a composition according to the invention.

Reference can be made—to avoid unnecessary repetitions—to above statements on the remaining aspects of the invention, which apply accordingly with reference to the inhalation stick according to the invention, for further details of this aspect of the invention.

Further features, advantages, and possible applications of the present invention result from the following description of preferred embodiments on the basis of the drawing and the drawing itself. In this case, all features which are described and/or illustrated in the figures form the subject matter of the present invention per se or in any arbitrary combination, independently of the summary thereof in the claims or what they refer to.

In the figures:

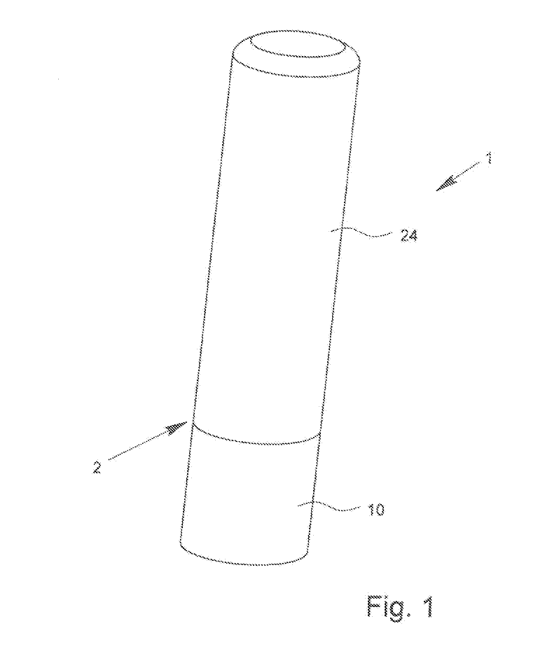

FIG. 1 shows a perspective view of an inhalation stick according to the invention,

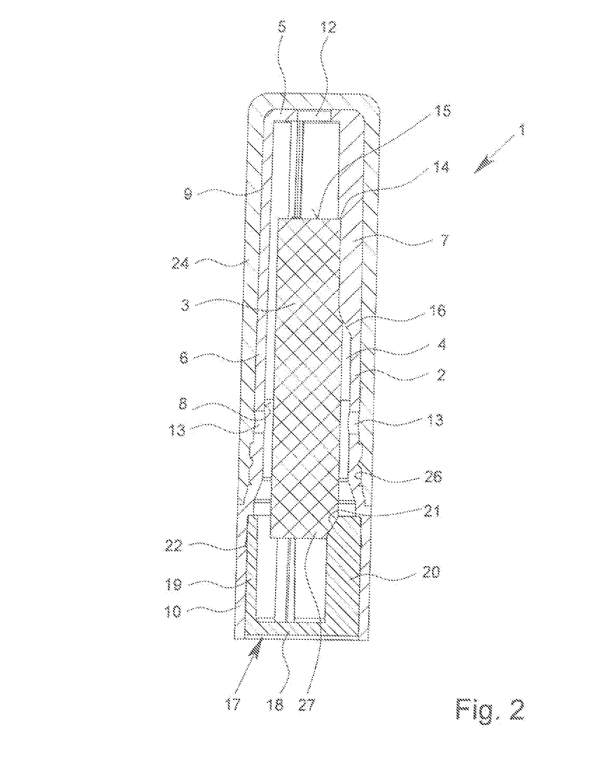

FIG. 2 shows a sectional view of the inhalation stick from FIG. 1,

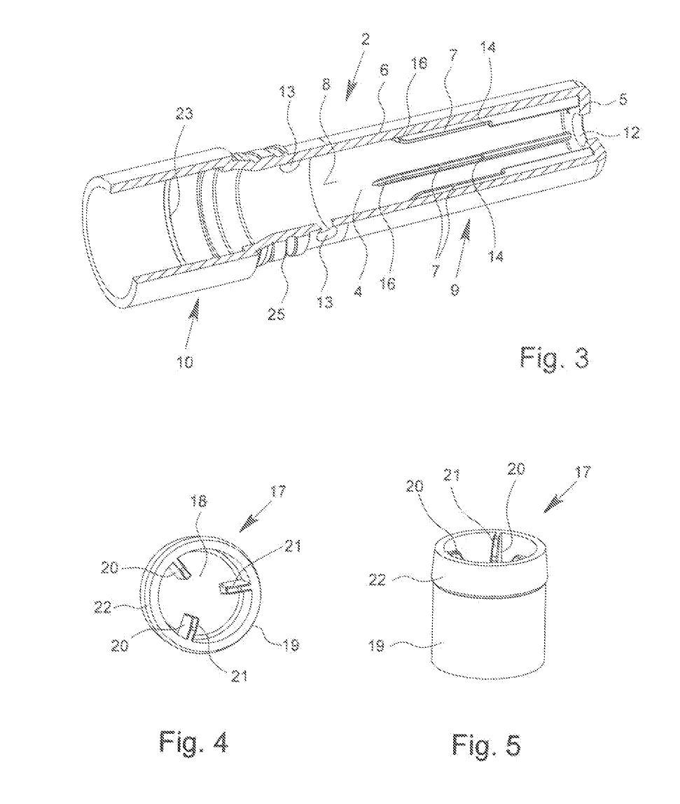

FIG. 3 shows a perspective sectional illustration of a housing sleeve of an inhalation stick according to the invention,

FIG. 4 shows a perspective view of a plug sleeve of the inhalation stick according to the invention,

FIG. 5 shows a further perspective view of the plug sleeve from FIG. 4, and

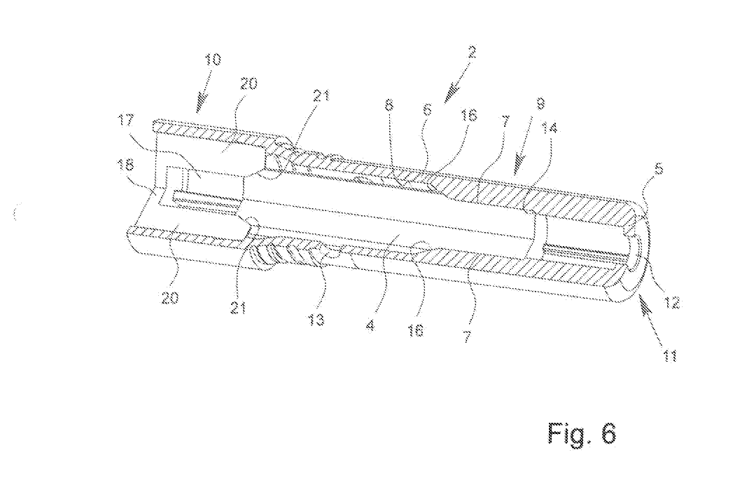

FIG. 6 shows a perspective sectional illustration of the inhalation stick according to the invention in the closed state without storage means.

FIGS. 1 and 2 show an inhalation stick 1. The inhalation stick 1 has an elongated housing sleeve 2 and rod-shaped storage means 3 made of a compressible material. The housing sleeve 2 consists in the present case of a plastic material. The storage means 3 is used for storing a preferably volatile liquid, which comprises at least one active ingredient. It is an essential oil in particular in this case. This liquid is also referred to as an inhalation or inhaling liquid. The storage means 3 itself can be a textile material, such as a nonwoven material. However, cotton materials can fundamentally also be used.

The storage means 3 has, in the described exemplary embodiment, an absorption capacity for inhalation liquid of at least 3 g/g, in particular at least 4 g/g, preferably at least 5 g/g. In the exemplary embodiment shown in FIG. 2, the storage means 3 has a length of 38 mm. The diameter can be between 1 and 20 mm, in particular between 6 and 14 mm, preferably between 8 and 12 mm depending on the type of the storage means 3.

To accommodate the storage means 3, the housing sleeve 2 has an inner receptacle space 4, which is delimited on the end by the end wall 5 and on the circumference by the outer wall 6.

It is now provided that at least one fixing projection, which protrudes in the radial direction into the receptacle space 4, is provided on the inside in the housing sleeve 2. In the illustrated exemplary embodiment, three elongated ribs 7 are provided in the housing sleeve 2, which are spaced apart equally from one another and are used as fixing projections. As shown in FIG. 2 in particular, the storage means 3 presses exclusively against the ribs 7, without coming into contact with the remaining inner wall region 8 of the wall 6. Friction-locked fixing of the storage means 3 results in the region of the mutual contact surface between the ribs 7 and the storage means 3. Because there is freedom from contact between the inner wall region 8 of the housing sleeve 2 and the storage means 3, diffusion of the inhalation liquid absorbed in the storage means 3 is not possible in this region. Because the ribs 7 are otherwise embodied as structurally identical, i.e., each protrude an equal distance from the wall 6, a centered arrangement of the storage means 3 in the receptacle space 4 results. As also shown in FIG. 2, the storage means 3 is compressed in the mutual contact region with the ribs 7, which promotes the friction lock between the ribs 7 and the storage means 3.

The housing sleeve 2 itself has a front sleeve region 9 and a rear sleeve region 10. The front sleeve region 9 is used as an inhalation region for the user. This means that the front sleeve region 9, its front end in any case, is provided for the insertion into or guiding to the nose of the user. For this purpose, the front sleeve region 9 has a central nose opening 12, which leads into the receptacle space 4, on the front end 11. Specifically, the nose opening 12 is provided in the end wall 5. In addition, at least one lateral opening 13 is located in the region of the rear end of the front sleeve region 9. In the illustrated embodiment, two lateral openings 13 are provided, which are located on opposing sides of the wall 6. The lateral openings 13 lead radially into the receptacle space 4 in the illustrated embodiment.

As results from various figures, the ribs 7 are provided in the region of the front end 11 of the front sleeve region 9. Finally, the ribs 7 extend in the illustrated embodiment from the end wall 5 in the axial direction into the receptacle space 4. Each of the ribs 7 has a stop 14 in this case for contact of the front end side 15 of the storage means 3. Furthermore, each of the ribs 7 has an intake bevel 16, which promotes the insertion of the storage means 3 into the housing sleeve 2.

A plug sleeve 17, which is inserted into the rear sleeve region 10, is provided for the closure of the housing sleeve 2. The plug sleeve 17, which also consists of plastic, is shown per se in FIGS. 4 and 5. The plug sleeve 17 has an outer end wall 18 and a cylindrical sleeve region 19. Fixing projections, which are again formed as ribs 20 in the illustrated exemplary embodiment, are also located inside the cylindrical sleeve region 19. The ribs 20 have terminal intake stops 21. As results in particular from FIG. 2, the storage means 3 is compressed in the region of the intake stops 21 in the final assembly state.

It is apparent that other embodiments of fixing projections can fundamentally also be provided instead of the ribs 7, 20. This is true in particular for the ribs 20. The number of the ribs 7, 20 provided in each case, which is three in each case in the illustrated exemplary embodiment, can also vary.

A latch is provided for connecting the plug sleeve 17 to the housing sleeve 2. A circumferential catch projection 22 is provided for this purpose on the outside of the cylindrical sleeve region 19 of the plug sleeve 17. A circumferential catch step 23 on the inside of the rear sleeve region 10 corresponds to the catch projection 22.

A closure cap 24, which can be put on the front sleeve region 9, is provided for the closure of the front sleeve region 9 having the sleeve opening 12 and the lateral openings 13. In the put-on state, the openings 12, 13 are covered and closed via the closure cap 24. Moreover, air-tightness results in the closed state, so that no escape of inhalation liquid results.

In the illustrated exemplary embodiment, a screw connection is provided for the secure hold of the closure cap 24 on the front sleeve region 9 and for ensuring sufficient leak-tightness between the closure cap 24 and the sleeve housing 2. For this purpose, an external thread 25, which corresponds to an internal thread 26 on the closure cap 24, is located at the transition from the front sleeve region 9 to the rear sleeve region 10. The screw connection or the thread pitch of the screw connection is formed so that opening of the closure cap 24 results upon a rotation of the closure cap 24 by less than 360°, preferably between 240° and 330°.

Moreover, it is apparent that it is fundamentally also possible to provide an easily detachable catch connection between the closure cap 24 and the housing sleeve 2 instead of a screw connection.

The assembly, including the filling of the inhalation stick 1, is performed such that firstly the storage means 3 is inserted into the housing sleeve 2. The insertion of the storage means 3 into the housing sleeve 2 can be performed at most enough that the storage means 3 stops with the front end side 15 on the stop 14. In this state, the storage means 3 is uncharged. Subsequently, the receptacle space 4 is closed by inserting the plug sleeve 17 into the rear sleeve region 10. In this case, the plug sleeve 17 is pressed into the outer sleeve region 10 until latching takes occurs between the catch projection 22 and the catch step 23. During the insertion of the plug sleeve 17 into the rear sleeve region 10, the intake stops 21 come into contact with the rear ends 27 of the storage means 3. In this case, the storage means 3 is compressed in the contact region in the still uncharged state. If the storage means 3 is not yet in contact with the stops 14 after the preliminary insertion, such a contact thus results at latest at this point in time. After latching of the plug sleeve 17, fixing of the storage means 3 in the axial direction results at six points, namely at the three stop points 14 and at the three points of the intake stops 21, wherein the storage means 3 is slightly clamped and therefore fixed.

Subsequently, the housing sleeve 2, which is provided with the storage means 3 and closed via the plug sleeve 17, is charged with the inhalation liquid. This is performed automatically via the nose opening 12. For this purpose, a filling needle is introduced into the centrally-fixed storage means 3. The introduction can be performed in this case such that the filling needle is led into the lower region of the storage means 3 and inhalation liquid is discharged continuously in steps during the subsequent removal or withdrawal. In this manner, optimum charging of the storage means 3 can be ensured over its entire length. It is apparent that it is fundamentally also possible, of course, that the filling needle is only inserted into the upper region of the storage means 3 and then inhalation liquid is discharged over a predefined time, this liquid subsequently being absorbed by the storage means 3.

The above-described types of charging the storage means 3 have proven to be outstandingly good, since a very hygienic and sterile situation results due to the charging of the storage means 3 in the already assembled state. In addition, very exact dosing of the inhalation liquid is possible.

In addition, the present invention—according to a fourth aspect of the present invention—relates to a method for assembling an inhalation stick of the above-mentioned type, wherein the storage means is firstly inserted into the receptacle space, the receptacle space is subsequently closed, and the inhalation liquid is introduced into the storage means via the nose opening after the closing of the receptacle space.

As already stated above, the inhalation stick may be charged very simply and cost-effectively with a precisely specified, defined dosing in the above-mentioned manner, wherein the charging finally takes place on location, so that sterility problems do not occur.

In addition, it has proven to be advantageous in conjunction with the filling of the inhalation stick if a partial vacuum or a vacuum is applied at least during the filling process or during the introduction of the inhalation liquid into the storage means, in particular at least in the sleeve. The introduced active ingredient solution (inhalation solution), in particular the preferably volatile liquid or the composition according to the present invention, then does not have to firstly displace the air located in the sleeve, so that the filling or production efficiency can be increased overall.

For more extensive details on this aspect of the invention—to avoid unnecessary repetitions—reference can be made to the above statements on the remaining aspects of the invention, which apply accordingly with reference to the method according to the invention.

Furthermore, the present invention—according to a fifth aspect of the present invention—relates to a further method for assembling an inhalation stick of the above-mentioned type, in which the above-mentioned method can fundamentally also be implemented, but does not have to be implemented. This method is also distinguished in that the storage means is firstly inserted into the receptacle space in the uncharged state, the receptacle space is subsequently closed by inserting the plug sleeve into the outer sleeve region, and the storage means is fixed at both ends in the longitudinal direction in its final assembly location due to the previously inserted plug sleeve. In this case, the dimensions of the length of the storage means, on the one hand, and/or the clearance between the fixing points of the housing sleeve and the plug sleeve, on the other hand, are finally such that either freedom from play, i.e., direct contact, results, or the storage medium is clamped with slight pressure at its ends. Moreover, this type of assembly enables the final assembly to the storage medium to finally take place via the insertion of the plug sleeve. In this manner, a defined arrangement of the storage means in the housing sleeve is ensured in any case.

For more extensive details on this aspect of the invention—to avoid unnecessary repetitions—reference can be made to the above statements on the remaining aspects of the invention, which apply accordingly with reference to the method according to the invention.

Finally, the present invention—according to a sixth aspect of the present invention—relates to the use of a composition as defined or described above for the nontherapeutic, in particular symptomatic application, in particular for application as a food, food ingredient or additive, nutritional supplement, flavor composition, or the like, in the case of illnesses from the group of cardiovascular illnesses (illnesses of the heart/circulatory system), in particular high blood pressure (hypertonia); infections, in particular colds, rhinitis, and bronchitis; metabolic illnesses, in particular diabetes or obesity; psychosomatic illnesses, in particular stress, physical-mental strain, withdrawal syndromes, and sleep disturbances, and/or chronic pain.

Nontherapeutic, in particular symptomatic application according to the present invention refers in particular to a use of the above-described composition according to the present invention, which is intended, in the case of a person suffering from one of the above-mentioned illnesses, not for healing or remedy in the medical or therapeutic meaning, but nonetheless is intended for at least subjective improvement of the well-being and/or the mental state of the affected person. For this purpose, the composition used can be provided or used as a food, food ingredient or additive, nutritional supplement, laboring composition, or the like, preferably by means of the above-described inhalation stick.

For more extensive details on this aspect of the invention—to avoid unnecessary repetitions—reference can be made to the above statements on the remaining aspects of the invention, which apply accordingly with reference to the use according to the invention.

Further embodiments, alterations, and variations as well as advantages of the present invention are readily recognizable and implementable for a person skilled in the art upon reading the description, without leaving the scope of the present invention in this case.

The present invention will be illustrated on the basis of the following exemplary embodiments, which do not restrict the present invention in any way, however.

### LIST OF REFERENCE NUMERALS

1 inhalation stick
2 housing sleeve
3 storage means
4 receptacle space
5 end wall
6 wall
7 rib
8 inner wall region
9 front sleeve region
10 rear sleeve region
11 front end
12 nose opening
13 lateral opening
14 stop
15 end side
16 intake bevel
17 plug sleeve
18 end wall
19 cylindrical sleeve region
20 rib
21 intake bevel
22 catch projection
23 catch step
24 closure cap
25 external thread
26 internal thread
27 rear end

### Examples

The influence of various inhalation sticks according to the invention was studied with reference to different medical and/or psychosomatic indications in the scope of the application and efficacy studies carried out by the applicant.

An inhalation stick according to the invention, as was described above and in particular also shown in FIGS. 1 to 6, was used for carrying out the following application studies.

Cellulose filters or cellulose acetate filters having a size of 38 mm length and 8 mm thickness, which are available from IVF Hartmann AG, Neuhausen, Switzerland or Filtrona Porous Technologies, Yverdon-les-bains, Switzerland, were used as the storage means for the respective employed composition according to the invention. The storage means in the inhalation sticks according to the invention were charged according to the method according to the invention in this regard with 0.8 mL of the compositions according to the invention in each case.

1. Inhalation Stick with an Aromatic Oil Combination of Lavender Oil, Vetiver Oil, and Mandarin Oil
The inhalation stick according to the invention contained, for test purposes, 0.8 mL of a composition according to the invention, which contained 35 wt. % lavender oil, 35 wt. % vetiver oil, and 30 wt. % mandarin oil, each in relation to the active ingredient composition. In the scope of the application studies carried out by the applicant, the influence of this combination according to the invention of essential oils on the heart/circulatory system of individuals who suffer from chronic stress was studied. An inhalation stick according to the invention without essential oils (“placebo stick”) was used as a comparison. In addition, it was ascertained for comparative purposes how strong the influence of the scientifically recognized relaxation technology of progressive muscle relaxation according to Jacobson is on the heart/circulatory system.
For this purpose, a test subject group of 90 persons, of whom 66 were female and 24 were male, having an age in the range from 18 to 65 years was used. The test subjects were included in the study under the condition that they fulfilled the criterion of an elevated chronic stress level. So as not to corrupt the obtained results, medical-clinical diagnoses, such as blood pressure illnesses, cardiovascular illnesses, diabetes, drug or medication dependency, epileptic attacks, pregnancy, mental illnesses, and acute colds, were excluded in the test subjects.
The study design was based on a placebo-controlled, randomized, three-armed exploration study with measurement repetition. The test subjects were allocated to the comparison groups listed hereafter in Table 1, each having 30 test subjects:

TABLE 1
experimental conditions, instructions, and tested effects

| group | instruction | tested effect |
|---|---|---|
| control/placebo | inhalation stick without | exclusion of nonspecific |
| group | active ingredient | effects (familiarity, |
| | | relaxation) |
| progressive | scientifically recognized | comparison of the achieved |
| muscle | calming and relaxation | physiological and |
| relaxation (PMR) | method | psychological relaxation |
| according to | | for the indication of |
| Jacobson | | chronic stress |
| inhalation stick | aromatherapy based on | study of the aromatic |
| | the active ingredient | substance effect with |
| | combination of lavender | reference to the |
| | oil, vetiver oil, and | physiological and |
| | mandarin oil | psychological relaxation |
| | | for the indication of |
| | | chronic stress |

Study Sequence
The test subjects were each individually tested, wherein the study duration was 45 minutes in total. The study began with the reception of the test subjects at point in time t₀.
After 12 minutes, at the point in time t₁₂, the sample subjects judged their present mental state or their well-being on the basis of a questionnaire to be filled out. The questionnaire contained 20 adjectives relating to mental state or well-being, comprising both positive and also negative effects, wherein the test subjects judged on a four-step scale whether the respective additive applied “not at all”, “hardly”, “somewhat”, or “very much”. Values in the range from 20 to 80 could be achieved in the scope of the judgment, wherein “80” corresponded to the most positive well-being and “20” to the most negative well-being.
Subsequently, the blood pressure and the heart rate were determined after 15 to 20 minutes with the aid of automatic oscillometric upper arm measuring devices (Hartmann, Heidenheim, Germany).
25 minutes after beginning the study, the 10 minute intervention was carried out at the point in time t₂₅₋₃₅. As described below, the intervention differed depending on the test subject group:

a) Control Group (Comparison)
The intervention in the control group was that the test subjects received the placebo inhalation sticks, which were identical with respect to the shape and size thereof to the inhalation sticks according to the invention, but did not contain any aromatic substances. The placebo inhalation stick was administered three times with an interstimulus interval of three minutes.

b) Group “Progressive Muscle Relaxation (PMR) According to Jacobson” (Comparison)
During the intervention, the test subjects were instructed to sequentially tension and then release specific body muscles step-by-step. The scheme took place in this case “from bottom to top”. In this case, thighs, buttocks, back, arms, and shoulders were alternately tensioned, and the actual relaxation was then carried out, i.e., all above-mentioned muscle parts were simultaneously tensioned for 10 seconds each. Toward the end of each tensioning, the test subjects were encouraged to intensify the tension. Overall, the test subjects went through three tensioning cycles with a two-minute interstimulus interval in each case.

c) Group Inhalation Stick with Aromatic Oil Composition (Invention)
The application of the inhalation stick according to the invention with the composition according to the invention was performed while recumbent in a relaxed body position. The test subjects placed the inhalation stick under one nostril and held the other nostril closed with a finger. In this case, the test subjects inhaled deeply once, wherein the composition according to the invention or the aromatic materials contained therein were held for a short time in the nose and subsequently exhaled again through the mouth. The inhalation stick was administered three times with an interstimulus interval of 3 minutes.
After completion of the intervention, a measurement of blood pressure and heart rate was performed again at the points in time t₃₆₋₄₁. At the point in time t₄₂, the subjectively perceived mental state of the test subject was determined again, in that the questionnaire was filled out a second time. After 45 minutes, at the point in time t₄₅, the study was terminated.

Results
The ascertained study results are set forth hereafter with reference to the mental state, the heart rate, and the blood pressure before and after the respective intervention of all test subject groups.
The mean values obtained in the individual groups and the standard deviation in this regard are specified in each case. The results of the measurement of the systolic blood pressure can be inferred from following Table 2, the results of the measurement of the diastolic blood pressure can be inferred from Table 3, the measured values of the heart rate can be inferred from Table 4, and the measured values for the subjective judgment of the mental state can be inferred from Table 5.

TABLE 2
mean values and standard deviations of the systolic blood pressure

| | mean value before | mean value after | difference |
|---|---|---|---|
| | [mmHg] | [mmHg] | [mmHg] |
| condition | (standard deviation) | (standard deviation) | (before/after) |
| control group | 119.13 | 117.38 | −1.75 |
| | (17.89) | (17.08) | |
| progressive | 123.47 | 116.33 | −7.14 |
| muscle | (18.26) | (17.8) | |
| relaxation | | | |
| inhalation stick | 126.04 | 114.18 | −11.86 |
| | (15.76) | (12.67) | |

With regard to the systolic blood pressure, the effect was greatest in the test subject group which had received the inhalation stick. The systolic blood pressure had sunk by an average of 11.86 mmHg after the intervention. In contrast, no noticeable reduction of the blood pressure was recorded in the control group. The test subjects who had applied the scientific relaxation technique of progressive muscle relaxation according to Jacobson during the intervention did have a stronger blood pressure reduction in comparison to the control group, but the effect was significantly less in comparison to the test subject group which had applied the
   inhalation stick containing active ingredients.

TABLE 3
mean values and standard deviations of the diastolic blood pressure

| | mean value before | mean value after | |
|---|---|---|---|
| | [mmHg] | [mmHg] | difference |
| condition | (standard deviation) | (standard deviation) | (before/after) |
| control group | 74.44 | 73.58 | −0.86 |
| | (12.49) | (13.03) | |
| progressive | 79.83 | 73.56 | −6.24 |
| muscle | (12.94) | (12.03) | |
| relaxation | | | |
| inhalation stick | 80.09 | 72.09 | −8 |
| | (13.06) | (10.33) | |

The effect was also greatest in the test subject group who had received the inhalation stick with reference to the reduction of the diastolic blood pressure. In contrast, the diastolic blood pressure was hardly able to be reduced in the control group. A moderate reduction of the diastolic blood pressure was recorded in the test subject group which had practiced progressive muscle relaxation.

TABLE 4
mean values and standard deviations of the heart rate

| | mean value before | mean value after | |
|---|---|---|---|
| | [mmHg] | [mmHg] | difference |
| condition | (standard deviation) | (standard deviation) | (before/after) |
| control group | 71.36 | 68.64 | −2.72 |
| | (11.43) | (11.31) | |
| progressive | 71.18 | 66.13 | −5.64 |
| muscle | (12.72) | (8.69) | |
| relaxation | | | |
| inhalation stick | 68.38 | 62.27 | −6.11 |
| | (12.47) | (10.92) | |

Significant differences were also able to be recorded with respect to the heart rate of control group from the test subject group which had been administered the inhalation stick and the test subjects who had practiced progressive muscle relaxation. The greatest effect had again occurred in the test subject group having the inhalation stick. In contrast, the smallest reduction of the heart rate was recorded in the control group.

TABLE 5
mean values and standard deviations of the mental state

| | mean value before | mean value after | difference |
|---|---|---|---|
| condition | (standard deviation) | (standard deviation) | (before/after) |
| control group | 54.2 | 57.67 | 3.47 |
| | (9.2) | (8.09) | |
| progressive | 57.27 | 61.27 | 4 |
| muscle | (7.19) | (6.61) | |
| relaxation | | | |
| inhalation stick | 55.33 | 66.07 | 10.74 |
| | (8.59) | (7.55) | |

Surprisingly, it was also shown with respect to the mental state that in the test subject groups which had applied the inhalation stick, the mental state or the well-being was able to be significantly increased already after a short intervention interval of 10 minutes or after three applications of the inhalation stick. It was particularly surprising in this context that the scientifically recognized relaxation method of progressive muscle relaxation also did not cause any noteworthy increase of the well-being, but rather the result was comparable to the control group. It was thus not expected that an increase of the well-being could be achieved using the inhalation stick according to the invention, which even exceeds scientifically recognized relaxation techniques.
Overall, significant differences before and after the experimental intervention between the individual groups therefore result in the analysis of the above-mentioned measured values. The best results were able to be achieved using an inhalation stick according to the invention, which contained a composition according to the invention based on lavender oil, vetiver oil, and mandarin oil, both with reference to physical factors, i.e., a reduction of blood pressure and heart rate, and also with reference to psychological factors, i.e., the increase of the well-being.
Inhalation sticks of the above-mentioned type are therefore outstandingly suitable to treat the symptoms accompanying chronic stress, of cardiovascular illnesses, such as high blood pressure and tachycardia, and also psychosomatic symptoms, such as fatigue, lethargy, and exhaustion, in a manner which has few side effects, preferably no side effects, and is additionally extremely comfortable to handle.
   2. Inhalation Stick with an Aromatic Oil Combination of Bergamot Oil, Lemon Oil, Cedarwood Oil, Clary Sage Oil, and Vetiver Oil
The inhalation stick according to the invention contained, for test purposes, 0.8 mL of a composition according to the invention, which comprised 34 wt. % bergamot oil, 28 wt. % lemon oil, 14 wt. % cedarwood oil, 14 wt. % clary sage oil, and 5 wt. % vetiver oil, each in relation to the active ingredient composition. In the scope of the application studies carried out by the applicant, the influence of this combination according to the invention of essential oils on the heart/circulatory system of individuals who suffer from chronic stress was studied. An inhalation stick according to the invention without essential oils (“placebo stick”) was used as a comparison. In addition, it was ascertained for comparative purposes how strongly the influence of the scientifically recognized relaxation technology of progressive muscle relaxation according to Jacobson is on the heart/circulatory system.
For this purpose, a test subject group of 45 persons, of whom 33 were female and 12 were male, having an age in the range from 18 to 65 years, was used. The test subjects were included in the study under the condition that they fulfilled the criterion of an elevated chronic stress level. So as not to corrupt the obtained results, medical-clinical diagnoses, such as blood pressure illnesses, cardiovascular illnesses, diabetes, drug or medication dependency, epileptic attacks, pregnancy, mental illnesses, and acute colds, were excluded in the test subjects.
The study design was based on a placebo-controlled, randomized, three-armed exploration study with measurement repetition. The test subjects were allocated to the comparison groups listed hereafter in Table 6, each having 15 test subjects:

TABLE 6
experimental conditions, instructions, and tested effects

| group | instruction | tested effect |
|---|---|---|
| control/placebo | inhalation stick without | exclusion of nonspecific |
| group | active ingredient | effects (familiarity, |
| | | relaxation) |
| progressive | scientifically recognized | comparison of the achieved |
| muscle | calming and relaxation | physiological and |
| relaxation (PMR) | method | psychological relaxation |
| according to | | for the indication of |
| Jacobson | | chronic stress |
| inhalation stick | aromatherapy based on | study of the aromatic |
| | the active ingredient | substance effect with |
| | combination of bergamot | reference to the |
| | oil, lemon oil, cedarwood | physiological and |
| | oil, clary sage oil and | psychological relaxation |
| | vetiver oil | for the indication of |
| | | chronic stress |

Study Sequence
The test subjects were each individually tested, wherein the study duration was 53 minutes in total. The study began with the reception of the test subjects at point in time t₀.
After 12 minutes, at the point in time t₁₂, the sample subjects judged their present mental state or their well-being on the basis of a questionnaire to be filled out. The questionnaire contained 20 adjectives relating to mental state or well-being, comprising both positive and also negative effects, wherein the test subjects judged on a four-step scale whether the respective additive applied “not at all”, “hardly”, “somewhat”, or “very much”. Values in the range from 20 to 80 could be achieved in the scope of the judgment, wherein “80” corresponded to the most positive well-being and “20” to the most negative well-being.
Subsequently, the blood pressure and the heart rate were determined after 15 to 20 minutes with the aid of automatic oscillometric upper arm measuring devices (Hartmann, Heidenheim, Germany). In addition, a saliva sample was taken to determine the cortisol level. Cortisol is a stress-associated hormone, which is excreted by the human body in stressful situations.
25 minutes after beginning the study, the 10 minute intervention was carried out at the point in time t₂₅₋₃₅. As described below, the intervention differed depending on the test subject group:

a) Control Group (Comparison)
The intervention in the control group was that the test subjects received the placebo inhalation sticks, which were identical with respect to the shape and size thereof to the inhalation sticks according to the invention, but did not contain any aromatic substances. The placebo inhalation stick was administered three times with an interstimulus interval of three minutes.

b) Group “Progressive Muscle Relaxation (PMR) According to Jacobson” (Comparison)
During the intervention, the test subjects were instructed to sequentially tension and then release specific body muscles step-by-step. The scheme took place in this case “from bottom to top”. In this case, thighs, buttocks, back, arms, and shoulders were alternately tensioned, and the actual relaxation was then carried out, i.e., all above-mentioned muscle parts were simultaneously tensioned for 10 seconds each. Toward the end of the tensioning, the test subjects were encouraged to intensify the tension. Overall, the test subjects went through three tensioning cycles with a two-minute interstimulus interval in each case.

c) Group Inhalation Stick with Aromatic Oil Composition (Invention)
The application of the inhalation stick according to the invention having the composition according to the invention was performed while recumbent in a relaxed body position. The test subjects placed the inhalation stick under one nostril and held the other nostril closed with a finger. In this case, the test subjects inhaled deeply once, wherein the composition according to the invention or the aromatic materials contained therein were held for a short time in the nose and subsequently exhaled again through the mouth. The inhalation stick was administered three times with an interstimulus interval of three minutes.
After completion of the intervention, a measurement of blood pressure and heart rate was performed again at the points in time t₃₉₋₄₄. At the point in time t₄₅₋₄₈, the subjectively perceived mental state of the test subject was determined again, in that the questionnaire was filled out a second time. Finally, the test subjects gave a second saliva sample at the point in time t₄₉₋₅₂. After 53 minutes, at the point in time t₅₃, the study was terminated.

Results
The ascertained study results are set forth hereafter with reference to the mental state, the heart rate, and the blood pressure before and after the respective intervention of all test subject groups.
The mean values obtained in the individual groups and the standard deviation in this regard are specified in each case. The results of the measurement of the systolic blood pressure can be inferred from following Table 7, the results of the measurement of the diastolic blood pressure can be inferred from Table 8, the measured values of the heart rate can be inferred from Table 9, and the measured values with respect to the determination of the cortisol level can be inferred from Table 10. Finally, Table 11 contains the measured values obtained for the subjective judgment of the mental state.

TABLE 7
mean values and standard deviations of the systolic blood pressure

| | mean value before | mean value after | difference |
|---|---|---|---|
| | [mmHg] | [mmHg] | [mmHg] |
| condition | (standard deviation) | (standard deviation) | (before/after) |
| control group | 117.07 | 117.29 | 0.22 |
| | (12.2) | (13.27) | |
| progressive | 117.64 | 114.51 | −3.13 |
| muscle | (11.73) | (12.98) | |
| relaxation | | | |
| inhalation stick | 122.98 | 108.49 | −14.49 |
| | (18.85) | (17.94) | |

With regard to the systolic blood pressure, the effect was greatest in the test subject group which had received the inhalation stick. The systolic blood pressure had sunk by an average of 14.49 mmHg after the intervention. In contrast, no noticeable reduction of the blood pressure was recorded in the control group. In the test subjects who had applied the scientific relaxation technique of progressive muscle relaxation according to Jacobson during the intervention, only a minor reduction of the systolic blood pressure also took place in comparison to the test subject group which had applied the inhalation stick containing active ingredients.

TABLE 8
mean values and standard deviations of the diastolic blood pressure

| | mean value before | mean value after | |
|---|---|---|---|
| | [mmHg] | [mmHg] | difference |
| condition | (standard deviation) | (standard deviation) | (before/after) |
| control group | 74.22 | 73.58 | −0.64 |
| | (8.55) | (9.19) | |
| progressive | 71.96 | 67.38 | −4.58 |
| muscle | (8.14) | (7.09) | |
| relaxation | | | |
| inhalation stick | 74.82 | 68.36 | −6.47 |
| | (10.4) | (10.69) | |

The effect was also greatest in the test subject group who had received the inhalation stick with reference to the reduction of the diastolic blood pressure. In contrast, the diastolic blood pressure was hardly able to be reduced in the control group. A moderate reduction of the diastolic blood pressure was observed in the test subject group which had practiced progressive muscle relaxation.

TABLE 9
mean values and standard deviations of the heart rate

| | mean value before | mean value after | |
|---|---|---|---|
| | [mmHg] | [mmHg] | difference |
| condition | (standard deviation) | (standard deviation) | (before/after) |
| control group | 73.56 | 72 | −1.56 |
| | (11.61) | (11.01) | |
| progressive | 74.67 | 71.82 | −2.85 |
| muscle | (15.93) | (14.86) | |
| relaxation | | | |
| inhalation stick | 72.09 | 64.64 | −7.45 |
| | (11.19) | (9.29) | |

Significant differences were also able to be recorded with respect to the heart rate of control group from the test subject group which had been administered the inhalation stick and the test subjects who had practiced progressive muscle relaxation. The greatest effect had again occurred in the test subject group having the inhalation stick. In contrast, the smallest reduction of the heart rate was recorded in the control group.

TABLE 10
mean values and standard deviations of the cortisol level

| | mean value before | mean value after | difference |
|---|---|---|---|
| condition | (standard deviation) | (standard deviation) | (before/after) |
| control group | 0.29 | 0.29 | 0 |
| | (0.12) | (0.13) | |
| progressive | 0.29 | 0.27 | −0.02 |
| muscle | (0.13) | (0.14) | |
| relaxation | | | |
| inhalation stick | 0.3 | 0.25 | −0.05 |
| | (0.17) | (0.14) | |

The study of the cortisol level also had the result that the application of the inhalation stick according to the invention has a positive effect on the alleviation of stress associated symptoms. The strongest reduction of the cortisol content in the saliva was able to be recorded in the test subject group which had applied the inhalation stick according to the invention with the aromatic oil composition. In contrast, hardly any reduction of the cortisol level was recorded in the control group. The reduction of the cortisol content was also significantly less in the test subject group which had applied progressive muscle relaxation than in the test subject group which had applied the inhalation stick according to the invention.

TABLE 11
mean values and standard deviations of the mental state

| | mean value before | mean value after | difference |
|---|---|---|---|
| condition | (standard deviation) | (standard deviation) | (before/after) |
| control group | 51.4 | 55.13 | 3.73 |
| | (8.16) | (8.13) | |
| progressive | 53.87 | 57.2 | 3.33 |
| muscle | (11.21) | (7.6) | |
| relaxation | | | |
| inhalation stick | 55.13 | 61.73 | 6.6 |
| | (8.05) | (5.28) | |

Surprisingly, it was also shown with respect to the mental state or the well-being that in the test subjects who had applied the inhalation stick, the mental state or the well-being was able to be significantly increased already after a short intervention interval of 10 minutes or after three applications of the inhalation stick. It was particularly surprising in this context that the scientifically recognized relaxation method of progressive muscle relaxation also did not cause any noteworthy increase of the well-being, but rather the result was comparable to the control group.
Overall, significant differences before and after the experimental intervention between the individual groups therefore result in the analysis of the above-mentioned measured values. The best results were able to be achieved using an inhalation stick according to the invention, which contained a composition according to the invention based on bergamot oil, lemon oil, cedarwood oil, clary sage oil, and vetiver oil, both with reference to physical factors, i.e., a reduction of blood pressure, heart rate, and cortisol level, and also with reference to psychological factors, i.e., the increase of the well-being. This would not have been predictable in this pronounced form.
Inhalation sticks with a composition according to the invention based on bergamot oil, lemon oil, cedarwood oil, clary sage oil, and vetiver oil are therefore outstandingly suitable to treat the symptoms accompanying chronic stress, of cardiovascular illnesses, such as high blood pressure and tachycardia, and also psychosomatic symptoms, such as fatigue, lethargy, and exhaustion, in a manner which has few side effects, preferably no side effects, and is additionally extremely comfortable to handle.
   3. Inhalation Stick with an Aromatic Oil Combination of Peppermint Oil and Grapefruit Oil
The inhalation stick according to the invention contained, for test purposes, 0.8 mL of a composition according to the invention, which contained 50 wt. % peppermint oil and 50 wt. % grapefruit oil, each in relation to the composition. In the scope of the application studies carried out by the applicant, the influence of this combination according to the invention of essential oils on weight reduction was studied. An inhalation stick according to the invention, which only contained lavender oil (“placebo stick”) was used as a comparison.
For this purpose, a test subject group of 40 persons was used, of whom 28 were female and 12 were male, having an age in the range from 24 to 61 years. The test subjects were permitted to join the study under the condition that their BMI (body mass index) was between 20 and 35. In addition, medical-clinical diagnoses, such as blood pressure illnesses, cardiovascular illnesses, diabetes, drug or medication dependency, epileptic attacks, pregnancy, mental illnesses, and acute colds, were excluded in the test subjects.

Study Design:
The study design was based on a placebo-controlled, randomized prospective documentation study. The test subjects were allocated into the comparison groups listed hereafter in Table 12, of 20 test subjects each:

TABLE 12
experimental conditions, instructions, and tested effects

| group | instruction | tested effect |
|---|---|---|
| control/placebo | inhalation stick with | nonspecific effects |
| group | lavender oil | (motivation, awareness) |
| inhalation stick | aromatherapy based on | study of the aromatic |
| | the active ingredient | material effect with |
| | combination of | reference to the |
| | peppermint oil and | assistance of weight |
| | grapefruit oil to | reduction (fat burning |
| | assist weight reduction | and appetite reduction) |

The study extended over a total period of time of 30 days, wherein the test subjects applied either the inhalation stick according to the invention or the placebo stick multiple times a day. The mode of application of the inhalation sticks was as follows in this case: all test subjects applied the respective inhalation stick to be tested 3 to 5 times approximately 5 to 10 minutes before each food intake. In addition, inhalation was additionally performed 3 to 5 times every two hours. So as not to influence the motivation or stress of the test subjects, they were not to weigh themselves at home during the study. In addition, the test subjects did not undertake any changes with reference to their remaining life rhythm or lifestyle, so as not to corrupt the effect of the inhalation sticks. In particular, the test subjects did not increase their sporting workload nor did they restrict their calorie supply or change their eating behavior.
To analyze the influence of the inhalation sticks on weight loss, the test subjects were weighed before beginning the study and after 30 days. In addition, the test subjects took part in a final survey, in the scope of which they were surveyed with reference to the perceived subjective effect of the inhalation sticks.

Results:
The ascertained study results with reference to the weight before and after the study of all test subjects are set forth hereafter. In addition, the results with reference to the final survey are explained in detail hereafter.
The results of the measurement of the weight of the test subjects at the beginning and at the end of the study can be inferred from following Table 13. In each case, the mean values obtained in the individual groups and the standard deviation in this regard are specified.

TABLE 13
mean values and standard deviations of the weight loss

| | average weight loss [kg] |
|---|---|
| condition | (standard deviation) |
| control group | 1.3 |
| | (0.5) |
| inhalation stick | 2.4 |
| | (0.3) |

As shown by the above statements, a significantly greater weight reduction could be achieved by the mere application of the inhalation stick according to the invention, without accompanying measures for weight reduction, for example, change in diet or more sport, than was the case for the placebo stick, which only contained lavender oil.
In addition, it was shown in the scope of the final survey that the test subjects who had applied the inhalation stick according to the invention had perceived less hunger during the period of time of the study and overall tended toward lower food intake. In the control group, who had lost significantly less weight on average than the test subject group with the inhalation stick according to the invention, significantly fewer test subjects indicated having had less hunger during the experimental period of time or having tended toward lower food intake.
Overall, it has been shown in the scope of the studies that the application of an inhalation stick according to the invention with the above-mentioned aromatic oil combination based on peppermint oil and grapefruit oil already resulted in weight reduction without additional assisting measures with respect to nutrition and sport. Therefore, inhalation sticks according to the invention can already be used alone for weight reduction, in addition, however, they also represent a reasonable supplement to diets and changes in diet.
   4. Inhalation Stick with an Aromatic Oil Combination of Peppermint Oil, Rosemary Oil, and Lavender Oil
The inhalation stick according to the invention contained, for test purposes, 0.8 mL of a composition according to the invention, which contained 60 wt. % peppermint oil, 30 wt. % rosemary oil, and 10 wt. % lavender oil, each in relation to the composition. In the scope of the application studies carried out by the applicant, the influence of the above-mentioned combination according to the invention of essential oils on menstrual pains and complaints of women.
A test subject group of 30 women having an age from 18 to 50 years was used for this purpose. The test subjects were permitted to join the study under the condition that they had regular menses and suffered from at least strong menstruation complaints. In addition, they could not be taking any prescription medication for reducing the monthly pains. Nonprescription medications, such as over-the-counter analgesics, were permitted, in contrast.

Study Sequence:
The study design provided that the test subjects observed their pains or complaints over to menstruation cycles, but only applied the inhalation stick according to the invention during one cycle. In addition, the test subjects could make use of their typical measures for alleviating complaints as needed during both cycles. The inhalation stick according to the invention could therefore be used more or less as an add-on for alleviating pain and complaints.
The application of the inhalation stick was performed every 10 minutes during the acute phase of the complaints or pains. The inhalation stick was inhaled deeply 10 times per application.
The test subjects were instructed to document the subjectively perceived pain intensity during the acute phases of the complaints and the mental state during both cycles and to compare the two cycles with one another.

Results:
It was shown with reference to the pain intensity that all test subjects were able to further alleviate the pains by way of the additional application of the inhalation stick according to the invention. In particular, it was shown in the scope of the studies that the application of the inhalation stick also had the effect that the other measures used, such as taking over-the-counter analgesics or heat therapies, can be significantly improved in their efficacy. In particular, it was shown in the scope of the study that the pharmacodynamics or pharmacokinetics of over-the-counter analgesics can be improved or accelerated, i.e., the latency, that is the time until the effect begins, and the pain duration until pain freedom can be significantly shortened. The mental state of the test subjects, in particular the general well-being, could also be significantly improved by the use of the inhalation stick according to the invention. The test subjects felt significantly more relaxed and better during their menstruation under application of the inhalation stick according to the invention.
Overall, it has therefore been shown in the scope of the application studies that the treatment of pains or painful complaints may be significantly improved by the use of inhalation sticks according to the invention. In particular, the inhalation stick according to the invention is also suitable as an assisting measure for accelerating the effect of over-the-counter analgesics for treating pains or chronic pains, for example, as occur in the scope of menstruation complaints.

### 5. Studies on Further Compositions According to the Invention

The influence of further compositions according to the invention were studied with reference to the treatment of various illnesses in the scope of further studies. The inhalation sticks were also charged as described above with the respective compositions. Test subject groups who suffered under the illnesses or symptoms listed in each case also apply the respective composition as described above using the respective inhalation stick.

### a) Composition Based on Peppermint Oil, Rosemary Oil, and Lavender Oil for Pain Therapy

The compositions with which the inhalation sticks were equipped contained 60 wt. % peppermint oil, 30 wt. % rosemary oil, and 10 wt. % lavender oil. The pains or chronic pain of the test subjects could be significantly alleviated by application of the inhalation stick with the above-mentioned composition.

### b) Composition Based on Grapefruit Oil and Peppermint Oil for Weight Reduction

The compositions with which the inhalation sticks were equipped contained 50 wt. % grapefruit oil and 50 wt. % peppermint oil. By application of the inhalation stick having the above-mentioned composition over a period of time of 4 weeks with simultaneous balanced diet without following a strict diet program, the selected test subjects, whose BMI was in the range of 23 to 27, could reduce their weight on average by 8%, in relation to the starting weight. The application of an inhalation stick with a composition based on grapefruit oil and peppermint oil therefore represents an effective assisting measure for weight reduction or treating obesity.

### c) Composition Based on Eucalyptus Oil, Peppermint Oil, and Thyme Oil for Treating Colds

The compositions with which the inhalation sticks were equipped contained 80 wt. % eucalyptus oil, 10 wt. % peppermint oil, and 10 wt. % thyme oil. Test subjects who suffered from colds applied the inhalation stick multiple times a day according to their personal needs. By application of the inhalation stick, classical cold systems, such as swollen nasal mucosa, difficult nasal respiration, and inflammations in the mouth/throat area could be significantly alleviated. Overall, the inhalation stick helped with rapid reduction of the cold symptoms and rapid regeneration.

### d) Composition Based on Lemon Oil, Peppermint Oil, Rosemary Oil, Grapefruit Oil, Pepper Oil, and Basil Oil

The compositions with which the inhalation sticks were equipped contained 25 wt. % lemon oil, 22 wt. % peppermint oil, 22 wt. % rosemary oil, 19 wt. % grapefruit oil, 7 wt. % pepper oil, and 5 wt. % basil oil. The well-being of the test subjects could be increased overall by application of an inhalation stick with the above-mentioned composition. In particular, the application of the compositions contributed to an improvement of the internal tranquility.

### e) Composition Based on Peppermint Oil, Vetiver Oil, Juniper Berry Oil, and Thyme Oil

The compositions with which the inhalation sticks were equipped contained 35 wt. % peppermint oil, 30 wt. % vetiver oil, 30 wt. % juniper berry oil, and 5 wt. % thyme oil. The well-being of the test subjects could be increased overall by application of an inhalation stick with the above-mentioned composition. In particular, the application of the compositions contributed to an improvement of the internal tranquility.

### f) Composition Based on Peppermint Oil, Cypress Oil, Geranium Oil, and Gingergrass Oil

The compositions with which the inhalation sticks were equipped contained 48 wt. % peppermint oil, 34 wt. % cypress oil, 14 wt. % geranium oil, and 4 wt. % gingergrass oil. The well-being of the test subjects could be increased overall by application of an inhalation stick with the above-mentioned composition. In particular, the application of the compositions contributed to an improvement of the internal tranquility.

### g) Composition Based on Peppermint Oil, Rosemary Oil, and Cinnamon Bark Oil

The compositions with which the inhalation sticks were equipped contained 86 wt. % peppermint oil, 11 wt. % rosemary oil, and 3 wt. % cinnamon bark oil. The well-being of the test subjects could be increased overall by application of an inhalation stick with the above-mentioned composition. In addition, the systems of heart/circulatory illnesses and of chronic stress could be significantly alleviated.

### h) Composition Based on Peppermint Oil, Grapefruit Oil, and Cinnamon Bark Oil

The compositions with which the inhalation sticks were equipped contained 90 wt. % peppermint oil, 8 wt. % grapefruit oil, and 4 wt. % cinnamon bark oil. The well-being of the test subjects could be increased overall by application of an inhalation stick with the above-mentioned composition. In particular, the application of the compositions contributed to an increase of concentration and motivation.

### i) Composition Based on Bergamot Oil, Marjoram Oil, Lavender Oil, and Orange Oil

The compositions with which the inhalation sticks were equipped contained 40 wt. % bergamot oil, 20 wt. % marjoram oil, 20 wt. % lavender oil, and 20 wt. % orange oil. The well-being of the test subjects could be increased overall by application of an inhalation stick with the above-mentioned composition. In particular, the application of the compositions contributed to an increase of concentration and motivation.

### j) Composition Based on Rosemary Oil, Peppermint Oil, and Basil Oil

The compositions with which the inhalation sticks were equipped contained 49 wt. % rosemary oil, 45 wt. % peppermint oil, and 6 wt. % orange oil. The well-being of the test subjects could be increased overall by application of an inhalation stick with the above-mentioned composition. In particular, the application of the compositions contributed to an increase of concentration and motivation.

1-15. (canceled)

16. An inhalation stick for inhalation through the nose,
wherein the inhalation stick comprises an elongated housing sleeve and a rod shaped storage means for storing at least one essential oil, wherein the housing sleeve comprises an inner receptacle space for receiving the storage means,
wherein at least one fixing projection, which protrudes in particular in the radial direction into the receptacle space, is provided in the interior in the housing sleeve for the friction-locked fixing of the storage means in the housing sleeve,
wherein a plug sleeve, which is insertable into the rear sleeve region, is provided for closure of the housing sleeve and wherein fixing projections, which are provided with terminal intake stops and are formed as ribs, are provided in the plug sleeve for the terminal fixing of the storage means.

17. The inhalation stick according to claim 16,
wherein a plurality of fixing projections are provided in the housing sleeve.

18. The inhalation stick according to claim 16,
wherein the storage means exclusively presses against the fixing projections, without pressing against the remaining inner wall regions of the receptacle space.

19. The inhalation stick according to claim 16,
wherein the storage means is arranged centered in the receptacle space by fixing projections, which are distributed uniformly around the circumference and are formed as elongated ribs.

20. The inhalation stick according to claim 16,
wherein the housing sleeve has a front sleeve region formed as an inhalation region and a rear sleeve region formed as a handle region.

21. The inhalation stick according to claim 16,
wherein at least one nose opening, which leads into the receptacle space, is provided on the front end of the front sleeve region and wherein at least one lateral opening, which leads radially into the receptacle space, is provided on the front sleeve region.

22. The inhalation stick according to claim 16,
wherein the ribs are provided in the region of the front end of the front sleeve region and wherein the ribs each have a stop for the terminal contact and fixing of the storage means in the axial direction and a terminal intake bevel.

23. The inhalation stick according to claim 16,
wherein the plug sleeve is latched with the housing sleeve and wherein a circumferential catch projection is provided externally on the plug sleeve and a circumferential catch step is provided on the inside of the rear sleeve region.

24. The inhalation stick according to claim 16,
wherein a closure cap is provided to be put on the front sleeve region for closure of the nose and lateral openings and wherein a screw connection is provided at the transition from the front sleeve region to the rear sleeve region for screwing together the closure cap with the housing sleeve.

25. The inhalation stick according to claim 16,
wherein the inhalation stick additionally comprises a composition for use in the prophylactic or therapeutic treatment of illnesses, wherein the inhalation stick comprises in its interior a storage means, wherein the storage means is provided with the composition.

26. The inhalation stick according to claim 25,
wherein the composition comprises at least two essential oils different from one another, wherein the essential oils are selected from the group of the following oils:
bergamot oil (Citrus aurantium var. bergamia),
cedarwood oil (Cupressus funebris Endlicher),
cypress oil (Cupressus sempervirens),
eucalyptus oil (Eucalyptus globulus),
geranium oil (Pelargonium graveolens),
gingergrass oil (Cymbopogon martinii var. sofia),
lavender oil (Lavandula angustifolia),
marjoram oil (Origanum majorana),
mandarin oil (Citrus reticulata),
clary sage oil (Salvia sclarea),
orange oil (Citrus aurantium var. dulcis),
peppermint oil (Mentha piperita),
pepper oil (Piper nigrum),
rosemary oil (Rosmarinus officinalis),
thyme oil (Thymus serpyllum),
vetiver oil (Vetiveria zizanoides),
juniper berry oil (Juniperus communis L.),
cinnamon bark oil (Cinnamomum ceylanicum),
lemon oil (Citrus limon),
grapefruit oil (Citrus decumana),
basil oil (Ocimum basilicum),
spruce oil (Picea excelsa)
and the mixtures and combinations thereof.

27. The inhalation stick according to claim 25,
wherein the storage means is designed as porous and air-permeable.

28. The inhalation stick according to claim 25,
wherein the storage means comprises a textile material based on cotton, cellulose, or cellulose derivatives and the mixtures and combinations thereof.

29. The inhalation stick according to claim 25,
wherein the inhalation stick and/or the storage means contain(s) the composition in relative quantities of 0.1 to 300 wt. %, in relation to the storage means.

30. The inhalation stick according to claim 25,
wherein the composition is volatile and has a vapor pressure at 20° C. of greater than 20 mbar.

31. A method for assembling an inhalation stick as defined in claim 16,
wherein the storage means is firstly inserted into the receptacle space in the uncharged state, wherein the receptacle space is subsequently closed and wherein the liquid is then introduced into the storage means after the closing of the receptacle space via the nose opening and/or the lateral opening.

32. The method according to claim 31,
wherein a partial vacuum is applied at least in the housing sleeve and at least during the introduction of the liquid into the storage means.

33. The method according to claim 31,
wherein the storage means is firstly inserted in the uncharged state into the receptacle space, wherein the receptacle space is subsequently closed by inserting the plug sleeve into the rear sleeve region and wherein the storage means is then fixed at both ends in the longitudinal direction in its final installation location as a result of the inserted plug sleeve.
